(19) United States
(12) Patent Application Publication
(10) Pub. No.: US 2017/0137773 A1
(43) Pub. Date: May 18, 2017

(54) Cell strainer with functionalized mesh to separate biological components

(71) Applicant: Hams-Werner Heinrich, (US)

(72) Inventor: Hams-Werner Heinrich, (US)

(21) Appl. No.: 14/940,147

(22) Filed: Nov. 13, 2015

(51) Int. Cl. C12M 1/00

(52) U.S. Cl. CPC C12M 47/02

(57) ABSTRACT

A cell strainer based particle separation system is described consisting of a functionalized mesh and micro-particle smaller than the mesh size. The micro-particles carry at least two binding moieties, one binding on the mesh-fibers, and the second specific to the selected cells. The selected cells can be separated from the biological liquid, modified, detached, analyzed, cultivated or otherwise manipulated.

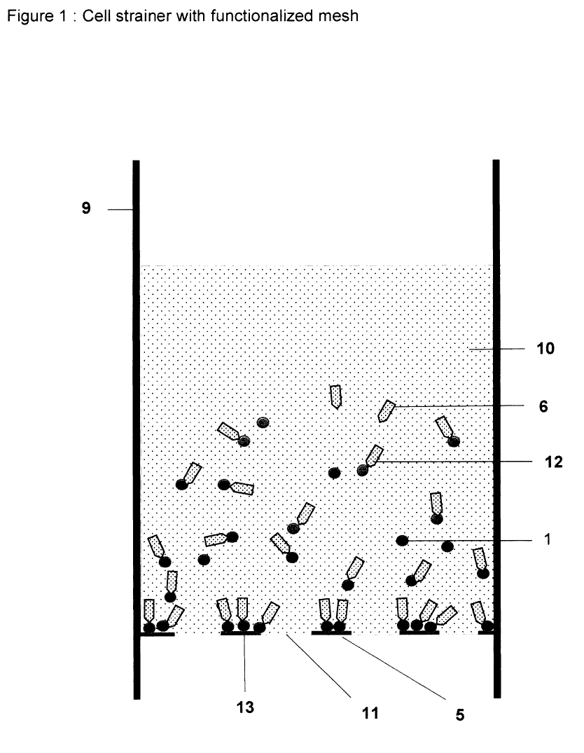

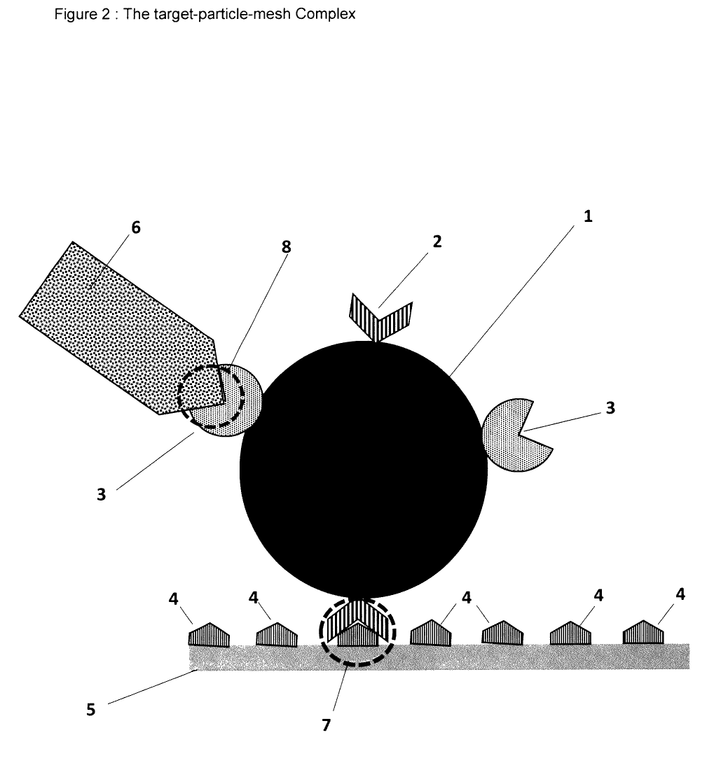

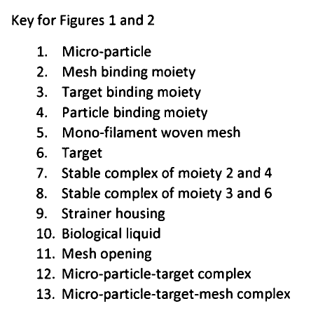

### FIELD OF THE INVENTION

This invention relates to a combination of micro-particle based immune-affinity system with the functionalized mesh-based strainer system for separating specific biological components from a suspension/dispersion of different biological origin.

### BACKGROUND OF THE INVENTION

The separation and isolation of biological components like specific pro- and eucaryotic cells, cell components, viruses or others from complex biological liquids like blood or other specimens is an ongoing challenge in all biological and medical research fields. Since the introduction of monoclonal antibodies, a tool is available to target the wanted component specifically. The immune-affinity chromatography was born in the 1950's by the coupling of antibodies to chromatography gels. Since then, this basic principle has being used in numberless applications including to isolate target cells from biological liquids. The principle is always the same. A specific binding moiety, typically an antibody, is coupled on a solid support. In most of the applications the solid support are particles or fibers. The cell suspension passes through a vessel (column) packed with the support. As the cells pass over the immobilized ligand-coated support, the ligand interacts with the specific molecule on the target cell surface, thus capturing the target of interest. Non-reactive cells/targets are washed out through the column. The captured targets were released by disrupting the bond between the ligand and its counterpart, resulting in isolating a homogenous population of cells/targets. The solid support is retained in the column by a mesh of openings size smaller than the solid support, typically of 20 μm (see also: Encyclopedia of Chromatography (Edt. J. Cazes), Third Edition, 2009; Volume II (Terry M. Phillips) Cells: Affinity Chromatography).

Different approaches to separate the anti-body targeted cells have been successful used. Magnetic forces can be used after introducing ferromagnetic characteristics in the specific binding moiety (U.S. Pat. Nos. 5,411,863; 5,759,793).
Differences in the specific weight of cells are successfully used in commercial cell separation systems based on gravity/centrifugation (U.S. Pat. No. 6,872,567; U.S. Pat. No. 6,211,315) or density gradient separation. Differences in particle size are basis for cell separation described in U.S. Pat. No. 8,815,092; U.S. Pat. No. 8,969,073.

The simplest way of target separation according particle size is the ancient method of sieving by means of strainers. WO 1993001271 describes a strainer hanging inside a vessel. The small size of the filter/mesh area in devices for laboratory use is the result of practical laboratorial work. Only a limited amount of sample material is needed, all necessary other buffers and disposables are minimized, rack space is reduced, more samples can be handled parallel, and, at the end, costs are reduced.
EP2664367A1 describes a cell strainer which is compatible with tubes of different opening sizes, i.e. fits on at least both the standard 15 ml and 50 ml laboratory tubes. The strainer consists of two parts, an “upper portion” which is the original strainer, and a “lower portion” which is an adaptor between the strainer and the standard tubes.
It is designed to fit into the openings of different sizes. The cell strainer according EP2664367A1 can be used for the removal of cell aggregates or large particles after tissue dissociation or from blood samples of up to 50 ml to obtain uniform single-cell suspensions. The design of the filters allows ventilation during filtration, and so avoids clogging of the filter.
The company Pluriselect (DE-Leipzig) introduced a non-magnetic, particle based target-separation system by using functionalized, target cell binding particles which are larger than the mesh size of the strainer. The separation principle is sorting according particle size. The mesh separates the target-particle-complex from the rest of the sample.

Nearly all described methods of cell separation use somewhere a mesh to separate the cells from the functionalized matrix or separation unit. All cell strainers have only one function, to hold particles mechanically back which are larger than the mesh size of the strainer.

The research community needs a cell/target separation system which combines the simplicity of a cell strainer with the principle of immune-affinity for target identification, modification, and separation. This cell separation system should cover sample volumes and target cell numbers which are common in research or diagnostics. Furthermore, the separation system should alleviate the disadvantage of commonly used polymer particles as solid immune affinity support which are in size distinctively larger than the target cells. They have to be larger than the cells, for the practical reasons of separation of the particles from the unwanted cells. The different size relation between polymer particle and cell results in limited excess of the ligands (e.g. membrane epitope) to the binding site of the antibodies coupled to the polymer particles. Cells with a high density of the targeted epitope expose enough ligands to the antibodies of the polymer particles. But for cells with a low expression of specific epitopes, like certain CD326 expressing carcinoma cells or CD56 on natural killer cells, these particle based systems fail. A sufficient separation of low expressing target cells is only possible if a large amount of available epitopes can be used as “leverage” for the separation principle. That is the case in using magnetized antibodies for magnetic cell separation. The disadvantage of this system is that all epitopes are blocked for further analytics and the antibodies get internalized and so do their ferromagnetic part.

A goal of the invention is to combine the advantage of easy to handle strainer system with micro-particles which are distinctively smaller as the target cell.
The present invention is generally related to isolate biological components. More specifically, the present invention provides systems, devices, and methods for the isolation, separation and/or processing of biological materials to culture and/or separate components from a biological liquid. Basis for the device and system according the invention are:
   1. cell strainer with a microfilament mesh which is functionalized to bind one moiety
   2. binding entity, preferably a micro-particle with at least two binding moieties, one part which binds to the filaments of the mesh and the second element binds the target cell/particle.

### SUMMARY OF THE INVENTION

The present invention reveals a system and a method to isolate, separate, modify and/or analyze targets, especially cells from samples of biological origin. The invention combines the simplicity of common strainers according to the invention consisting of functionalized microfilaments with the versatility of functionalized surfaces of micro-particles as immune-affinity unit binding the target cells.
This is achieved according to the invention, by functionalizing the micro-fibers of the woven mesh preferably with biotin or avidin/streptavidin or biotin-binding protein. And the usage of a binding entity, preferably a micro-particle carries at least two binding moieties, one part which binds to the filaments of the mesh and the second element binds the target cell/particle.
The strainer should be equipped with a valve system to create, for a certain period of time, a reaction compartment above the mesh. During this time the micro-particle target cells complex binds to the micro-fibers of the strainer mesh.
The targets can now be separated from the sample liquid by opening the outlet valve. A washing step is recommended for positive selection.

### DESCRIPTION OF THE INVENTION

The strainer mesh consists of precision woven monofilaments. Commonly used is polypropylene (PP), polyamide (PA), polyethylene terephthalate (PET), polyether ether ketone (PEEK) or Ethylene tetrafluoroethylene (ETFE). The mesh structure allows high flow rates. It is simple to handle. The smaller components of the sample and the biological liquid can be easily separated from particles larger than the mesh size. The mesh size can be chosen according to the particle size which has to be retained. The weaving technology and filament structure determines the open area of the mesh. Depending on the fiber material and the mesh size, the open area of the mesh is only between 1% and 50%, and the remaining area consists of the microfilaments.

According to the invention, the microfilaments are used as the carrier of any binding activity, preferably one partner out of the biotin-avidin/streptavidin binding system. According to the invention, the non-open area of mesh, 50%-99%, represents solid support and is used for functionalization preferably with biotin. It is the decisive component of the device and system. The biotin-avidin/streptavidin interaction is known to be one of the strongest non-covalent interactions with dissociation constant, Kd˜10-1

According to the invention, micro-particles are used which are distinctively smaller than the target cells and the mesh size of the strainer is larger than the cells. To create a versatile system the micro-particle must be equipped with a moiety which binds to the solid support of the mesh. At least a second moiety of the micro-particle must carry the specific ligand which binds to the target.
The strainer should be equipped with a unit to regulate the outflow down to zero. That creates for a certain period of time a reaction room above the mesh to allow the binding of the target-micro-particle-complex.
During the reaction time, the micro-particle forms a bridge between the target and the functionalized fibers of the mesh. The efficacy of the target separation depends on the size of the micro-particles, the density of the specific cell membrane ligands, the avidity of the binding partners, and the incubation condition.

Most preferable for target cell separation and processing are micro-particle sizes in the range of 5 nm up to 5 μm. The mesh-size for the cell separation is most preferable between 20 and 100 μm, which provides between 90% to 60% usable mesh area for the capturing and binding of the micro-particles. For other applications, different particle sizes and mesh sizes may be more useful.

For example, the open area of a round mesh with a diameter of 20 mm and mesh size of 30 μm is ca. 30%. The remaining 70% (or 2 cm²) of the mesh can be used to separate the targets. This area is sufficient to separate more than 10⁷ target cells of the size of mononuclear blood cells (PBMC), a target number which makes the simple-to-use device attractive for most biological and medical research fields and diagnostics.

The preferred geometry of the solid support is a mesh of precision woven monofilaments. Commonly used is polypropylene (PP), polyamide (PA), polyethylene terephthalate (PET), polyether ether ketone (PEEK) or Ethylene tetrafluoroethylene (ETFE). The mesh structure allows high flow rates and is easy to wash.

PET is the preferred material for functionalization. It is widely used in various biological applications as a biocompatible and hemocompatible material. However, PET must undergo a surface modification due to its low functionality. The surface of PET can be functionalized by using a variety of techniques, such as chemical treatment, UV irradiation, plasma treatment, ion implantation, etc. The chemical aminolysis of PET with polyethylenimine or ethylendiamine is one method to introduce free amino groups on their surface. The native PET fibers are aminated using polyethylenimine followed by cross-linking with 1,4-butanediol diglycidyl ether or simply by treating the fibers with ethylendiamine. The generated functional amino groups now provide the possibility for further chemical reaction, preferably to bind biotin or avidin/strepavidin. But it is not restricted to the two reaction partner. The created functional groups can be used to modify the micro-filaments of the mesh to achieve new qualities and applications.

N-Hydroxysulfosuccinimide (Sulfo-NHS) esters of biotin are the most popular type of biotinylation reagent. NHS-activated biotins react efficiently with primary amine groups (—NH2) in pH 7-9 buffers to form stable amide bonds. Several different NHS esters of biotin are available, with varying properties and spacer arm lengths. The sulfo-NHS ester reagents are water soluble, enabling reactions to be performed in the absence of organic solvents.

The use of mono-disperse micro-particles is state-of-the-art for many applications in research and diagnostics. The materials are mostly polymethly methacrylate, polystyrene, melamine resins, polydivinylbenzene, poly(styrene-co-divinylbenzene), poly(styrene-co-methacrylic acid methylester), mono disperse silica. They are available with many different surface modifications with functional groups NH2, epoxy, COOH, alkyl-OH, etc. with or without different spacer of different length or modified with protein, biotin, chitosan, PEG, etc. The size of mono-disperse micro-particle according to the invention can be between 1 nm and more than 100 μm, depending on the mesh size of the strainer and the specific application.

The most preferred micro-particle material is polystyrene with functional NH2 groups. The maleimide chemistry is most preferred for directed functionalization. Crosslinker like Sulfo-SMCC (sulfosuccinimidyl 4-[N-maleimidomethyl] cyclohexane-1-carboxylate) with maleimide can be used. The maleimide groups are introduced on the surface of aminated particles without or with spacers of different flexibility and length to achieve an optimal orientation of the conjugated biomolecules to preserve their biological activity. Sulfo-SMCC, a hetero-bifunctional crosslinker that contains N-hydroxysuccinimide (NHS) ester and maleimide groups, allowing covalent conjugation of amine- and sulfhydryl-containing molecules. NHS esters react with primary amines on the micro-particle to form amide bonds. The maleimides react with sulfhydryl groups to form stable thioether bonds. Maleimide functionalized particles are very efficient for the binding of thiolated bio-molecules.
Many other combinations of methods of activation and functionalization of the surfaces of the micro-filaments and the micro-particle are available and can be chosen to achieve the goal according to the invention.

The micro-particles are conjugated with at least two binding moieties, the streptavidin and the ligand which binds the target. This ligand is preferably an antibody or parts of it, but is not restricted to it. It can be any molecule which has the capacity to bind specifically to the target, like lectins, peptides, oligo-nucleotides, etc.
The streptavidin and antibody coupling to the maleimide functionalized micro-particles is carried out in one step. Natural streptavidin has no sulfhydryl group. The protein has to be thiolated or recombinant streptavidin thiol has to be used.

The thiolated proteins (mixture of antibodies and streptavidin) and maleimide functionalized particles are incubated at room temperature for 3 hours in PBS and another hour after adding a cystein solution. They are ready for use after washing.

The liquid of biological origin can be any watery solution, or suspension and emulsion with water as dispersion phase (blood, spinal fluid, se- and excretes, tissue culture samples, suspension of tissue cells, suspension of microorganism including virus, suspension from parasites and part of them, suspension from fungi and parts of them, suspension from plankton organisms etc.).

To start the method, the strainers outlet with the biotinylated PET mesh is closed. The compartment above the mesh can be used as a reaction room.

An embodiment is to incubate samples with the micro-particle for 5-30 min, depending on the expected number of targets. The micro-particles can be one pre-added component of the specimen collection container. The micro-particles bind on the targets. Depending on the ratio of the micro-particle to the available epitopes, the majority of the epitopes should be covered with micro-particles. After pre-incubation, the sample with the micro-particles is transferred into the biotin-mesh-strainer and is then incubated for 5-30 minutes. The streptavidin-moiety of the micro-particles binds to the biotin-mesh and fixates the target on the strainer mesh. After opening the strainer valve, the strainer can be used in its typical function to release and separate the biological liquid from the targets. As the result, the mesh is exclusively covert with the targets bound via a two-binding-component bridge to the fibers of the mesh.

Another embodiment is the pre-coating of the fiber mesh with the micro-particle, according to the invention. The outlet valve of the strainer is closed. The needed amount of functionalized micro-beads depends on particle size, the expected number of target cells and available micro-filament surface. After incubating for 5-15 minutes the micro-beads bind to the biotin-mesh. After the incubation with the micro-beads, a washing step can be introduced to remove the remaining unbound micro-particles. The sample is added and incubated for 5-30 minutes. The target specific moiety of the mesh-fixated micro-particles, preferably an antibody, binds to the targets within the sample. After opening the strainer valve, the strainer can be used in its typical function to release and separate the targets from the sample. Now the mesh is exclusively covert with the targets bound via the two-binding-component bridge to the fibers of the mesh. In this case only the membrane epitopes facing the mesh surface are blocked by the bridging micro-particle complex. Epitopes not facing the mesh are still available for further manipulation and cell analysis.

Both methods result in target cells fixated to the micro-filaments of the mesh. These immobilized cells can be used for further analysis which does not depend on cell integrity, like isolation of nucleic acids, proteins, cell organelles, etc.

Another embodiment is to expose the living immobilized cell to any kind of modifying agent, like antigen, proteins, soluble signal transmitter like cytokines and hormones, synthetic peptides, nucleic acids, vectors for gene modification or expression, oligo-nucleotides, pharmacologic substances including biologicals, chemicals, co-incubation with other cell population or microorganism including virus, and others. This can be done on top of the mesh after closing the outlet valve, or by placing the strainer in a cavity of a tissue culture plate.

Another embodiment is the use of cleavable spacers between the target specific binding moiety of the micro-bead and the bead. This allows the detachment of the targets from the micro-beads for further manipulation including cultivation.

Another embodiment is the use of ferromagnetic micro-particle for the method according to the invention. These particles with at least the two binding moieties can be separated from the target cells after their detachment from the mesh and the targets by the use of a magnet.

Although the two different moieties of the bridge system can just be formed by the two binding molecules, it is preferable to combine this complex with a spacer which can be cut by the particular detachment unit.

The binding bridge entity can be in the simplest form a streptavidin labeled antibody or other structure which specifically binds the target. For practical reasons of providing a research tool for innumerable targets, it is favorable to manufacture the e.g. uniform biotinylated strainers and supply the target specific bridge-systems separately.

The introduction of micro-particle as solid support for the bridge system is most favored. The micro-particles increase the usable surface of the complex mesh-fiber-micro-particle.

Another embodiment is combining particle separation, according to the invention, with using a functionalized particle larger than the mesh size to isolate and separate simultaneously different targets from the same sample.

1. A target separation device to separate and isolate targets from biological liquids consisting of a strainer with a functionalized mono-filament mesh and micro-particle of a size smaller than the mesh openings, carrying at least two binding capacities from which one moiety binds to the functionalized mesh material and the second one the target.

2. Device according 1, wherein the mesh material is functionalized with any reacting partner which creates with the second moiety on the micro-beads a stable complex.

3. Device according 1 and 2, wherein the binding partners are biotin-avidin/streptavidin, biotin-biotin binding protein, antibody-antigen or a single strand nucleic acids.

4. Device according to 1, wherein the moiety of the functionalized micro-particle which binds the target is an antibody, ligand, protein, peptide, lectin, lipid, or a single strand nucleic acid.

5. Device according 1, wherein the binding moieties of the micro-particles are equipped with a cleavable spacer between micro-particle and binding moiety.

6. Device according to 1, wherein the micro-particles have a size between 1 nm and 5 μm and the mesh openings have a size between 10 μm and 100 μm.

7. Device according 6, wherein the said micro-particles can comprise a ferromagnetic matrix.

8. Device according 1, wherein said the strainer outlet has a valve to generate a reaction compartment above the mesh.

9. Device according 1 to 8 for usage to separate targets from biological liquids for further manipulation

10. Device according 9, wherein the targets are cells.

11. Usage according 9 and 10, wherein said the manipulation includes detachment, incubation with a modifying agent, like antigen, proteins, soluble signal transmitter like cytokines and hormones, synthetic peptides, nucleic acids, vectors for gene modification or expression, oligo-nucleotides, pharmacologic substances including biologicals, chemicals, co-incubation with other cell population or microorganism including virus.
